(19) United States
(12) Patent Application Publication
(10) Pub. No.: US 2017/0151402 A1
(43) Pub. Date: Jun. 1, 2017

(54) RESPIRATORY THERAPY APPARATUS AND METHODS

(71) Applicant: SMITHS MEDICAL INTERNATIONAL LIMITED, Kent (GB)

(72) Inventors: Anthony Lucio Belisario, Luton (GB); Mohammad Qassim Mohammad Khasawneh, Luton (GB); Mark Charles Oliver, Hertfordshire (GB); Mark Sinclair Varney, Bedfordshire (GB)

(73) Assignee: SMITHS MEDICAL INTERNATIONAL LIMITED, Kent (GB)

(21) Appl. No.: 15/320,774

(22) PCT Filed: Jun. 5, 2015
(86) PCT No.: PCT/GB2015/000161

§ 371 Date: Dec. 21, 2016

(30) Foreign Application Priority Data
Jun. 24, 2014 (GB) 1411172.8

(51) Int. Cl. A61M 16/00

(52) U.S. Cl. CPC A61M 16/0072; A61M 16/0006; A61M 16/0009; A61M 2016/0027; A61M 2016/003; A61M 2205/3306

(57) ABSTRACT

Respiratory therapy apparatus has a short conduit (10) with a mouthpiece (13) at one end and open to atmosphere at its opposite end (11). One end of a cylinder (22), (110, 203) opens into the conduit (10) and contains a piston (21, 111, 205) slidable along the cylinder. The piston (21, 111, 205) carries a permanent magnet (24) that interacts with a magnetic field produced by electromagnetic coils (25, 26), 101-109 surrounding the cylinder. The coils are driven by a control unit (30) that receives inputs from pressure, flow and piston position sensors (40, 41) and (42) to cause the piston to oscillate in the cylinder and superimpose an oscillatory waveform on the normal tidal respiration along the conduit (10) at an amplitude sufficient to mobilize mucus in the patient's airway and produce a therapeutic effect.

This invention relates to respiratory therapy apparatus of the kind including a breathing system arranged to be coupled with a patient's airway.

Patients with respiratory system diseases, such as asthma, COPD, cystic fibrosis and the like, have a prominent pathophysiological feature in the form of hyper secretion of mucus, often accompanied by impaired mucus transport. This imbalance between mucus transport and secretion results in mucus being retained in the respiratory system. Positive expiratory pressure (PEP) apparatus, that is, apparatus that presents a resistance to expiration through the device, are now widely used to help treat patients suffering from a range of respiratory impairments. More recently, such apparatus that apply chest physiotherapy by providing an alternating resistance to flow have been found to be particularly effective. One example of such apparatus is sold under the trade mark Acapella (a registered trade mark of Smiths Medical) by Smiths Medical and is described in U.S. Pat. No. 6,581,598, U.S. Pat. No. 6,776,159, U.S. Pat. No. 7,059,324 and U.S. Pat. No. 7,699,054. Other vibratory respiratory therapy (V-PEP) apparatus is available, such as “Quake” manufactured by Thayer, “AeroPEP” manufactured by Monaghan, “TheraPEP” manufactured by Smiths Medical and “IPV Percussionator” manufactured by Percussionaire Corp. The generated vibratory positive pressures mechanically reduce the viscoelasticity of sputum by breaking down the bonds of mucus macromolecules which enhances mucociliary clearance. Alternative apparatus such as “CoughAssist” manufactured by Philips are also available. Respiratory therapy apparatus can instead provide an alternating resistance to flow during inhalation.

The input impedance of a respiratory system can be measured in the frequency band from sub-acoustic frequencies to around 50 Hz by imposing small amplitude waveforms onto the patient's airway. The resulting flows and pressure changes are recorded and used to calculate the real and imaginary parts of the input impedance. The waveforms imposed on the airway are of very small amplitude solely for measurement purposes and are insufficient to produce any therapeutic effect.

It is an object of the present invention to provide alternative respiratory therapy apparatus and methods.

According to one aspect of the present invention there is provided respiratory therapy apparatus of the above-specified kind, characterised in that the apparatus includes an arrangement coupled with the breathing system that superimposes an oscillatory waveform on normal tidal respiration via the breathing system at an amplitude sufficient to mobilise mucus in the airway and produce a therapeutic effect.

Preferably the waveform is asymmetric and arranged to have a greater peak flow in a direction out of the lungs than into the lungs so as to increase flow out of the lungs. The waveform is preferably a complex waveform constructed from a series of sinusoidal waveforms of differing periods. The arrangement coupled with the breathing system preferably includes a piston movable within a cylinder and may include a magnetic arrangement for moving the piston in an oscillating manner relative to the cylinder. Preferably, the piston includes a permanent magnet and the cylinder includes an electromagnetic coil arranged to produce a magnetic field within the cylinder that interacts with the field of the permanent magnet to displace the piston along the cylinder. The or each electromagnetic coil may extend around the outside of the cylinder. The apparatus preferably includes a position sensor responsive to the position of the piston. The breathing system preferably includes a conduit that is open to atmosphere at one end and opens to a mouthpiece at its opposite end. The apparatus preferably includes a pressure sensor responsive to pressure in the breathing system and a flow sensor responsive to flow in the breathing system. The apparatus is preferably arranged to adjust the nature of the generated waveform according to feedback from the breathing system.

According to another aspect of the present invention there is provided respiratory therapy apparatus having a conduit with a mouthpiece at one end and open to atmosphere at its opposite end, characterised in that one end of a cylinder opens into the conduit, that the cylinder contains a piston slidable along the cylinder, that the piston carries a permanent magnet that interacts with a magnetic field produced by at least one electromagnetic coil surrounding the cylinder, that the apparatus includes a control unit connected to the or each coil, that the control unit is connected to receive inputs indicative of the position of the piston in the cylinder and pressure in the conduit, and that the control unit is arranged to cause the piston to oscillate in the cylinder in response to the inputs thereby to superimpose an oscillatory waveform on the normal tidal respiration along the conduit at an amplitude sufficient to mobilise mucus in the patient's airway and produce a therapeutic effect.

According to a further aspect of the present invention there is provided a method of applying respiratory therapy to a patient's airway, characterised in that an oscillatory waveform is superimposed on normal tidal respiration at an amplitude sufficient to mobilise mucus in the airway and produce a therapeutic effect.

The waveform is preferably asymmetric and is arranged to produce a greater peak flow in a direction out of the lungs than into the lungs so as to increase flow out of the lungs. The waveform is preferably a complex waveform constructed from a series of sinusoidal waveforms of differing periods.

Respiratory therapy apparatus and its method of use will now be described, by way of example, with reference to the accompanying drawings, in which:

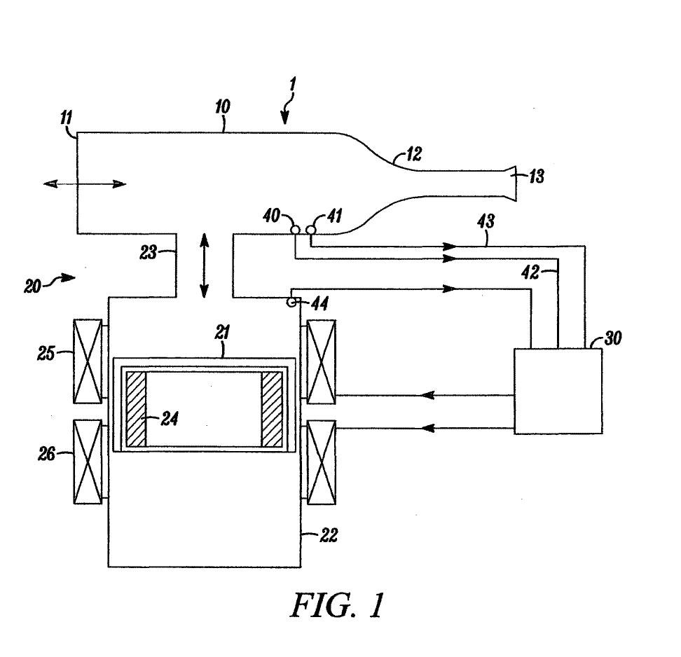

FIG. 1 illustrates the apparatus schematically;

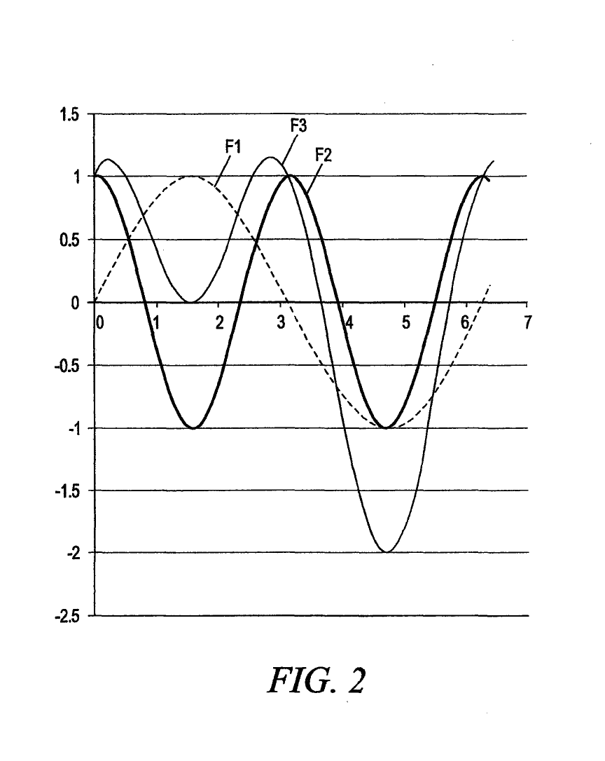

FIG. 2 is a graph showing the effect of combining two waveforms;

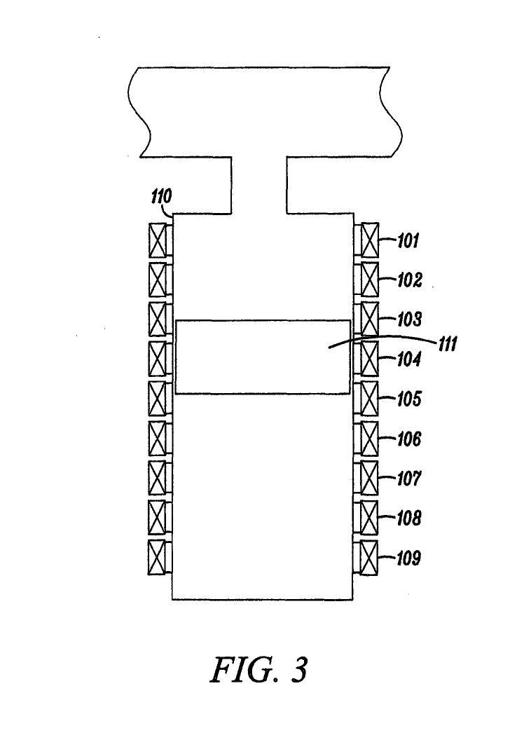

FIG. 3 shows schematically an alternative actuator arrangement; and

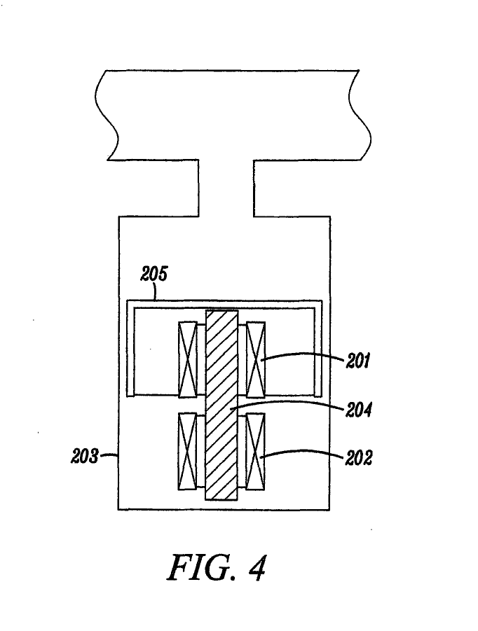

FIG. 4 shows schematically another alternative actuator arrangement.

With reference first to FIG. 1 the apparatus includes a breathing system 1 having a conduit 10 with one end 11 open to atmosphere and its opposite end 12 coupled with a breathing device in the form of a mouthpiece 13 so that air can flow to the facemask via the breathing system on demand from the patient. Other forms of breathing device could be used such as a tracheal tube, laryngeal mask or the like. The breathing system 1 could also include other components such as a humidifier, filter, nebulizer, a supply of supplementary oxygen or other gases. The breathing system 1 could also include means for modifying the flow of gases such as to provide a resistance to flow or a device to enhance flow.

The system also includes actuator means 20 coupled with the breathing system 1 and arranged to superimpose an oscillatory waveform on normal tidal respiration via the breathing system at an amplitude sufficient to mobilise mucus in the airway and produce a therapeutic effect. This means 20 coupled with the breathing system 1 is provided by a coil-wound actuator shown as including a piston 21 within a cylinder 22 that opens at one end 23 into the breathing conduit 10. The piston 21 includes a permanent magnet 24 of ring shape mounted coaxially within the piston. The cylinder 22 includes an arrangement of two wound electromagnetic coils 25 and 26 encircling the cylinder and spaced from one another along the length of the cylinder. The coils 25 and 26 are connected to a drive and control unit 30 arranged to energise the coils appropriately to set up a magnetic field within the cylinder 22 that interacts with the field from the permanent magnet 24 in the piston 21 in such a way that the piston is displaced along the length of the cylinder in an oscillatory manner. The piston 21 may be lubricated or have an exterior surface of a low friction material such as PTFE. The volume displaced by the piston 21 should preferably be about 500 ml. A filter (not shown) may be included between the cylinder 22 and the breathing system 1 to prevent any debris or contamination from the cylinder passing to the patient's airway and also to prevent the interior of the cylinder becoming contaminated by expired material from the patient. In this respect, interior surfaces of the apparatus could be coated with an anti-bacterial substance. The cylinder 22 may provide a convenient handle by which the patient can hold the apparatus up to his mouth where the apparatus is of the hand-held type although the apparatus could be incorporated into an in-line respiratory system.

Wound coil actuators are available in various sizes with a stroke length between 5 mm and 30 mm and a continuous force range between 2N and 70N with high peak forces. The actuators can have a low coil mass with a very fast response and high bandwidth. They can also have zero backlash, hysteresis and cogging, with no contact between the coil and core movement so there is no wear and tear. The actuators can also have a smooth motion at low speeds with limitless resolution, depending on the feedback mechanism. Various alternative electromagnetic arrangements are described later. It would also be possible to drive a piston along a cylinder using some other motive force such as, for example, provided by a piezoelectric actuator.

The apparatus also includes a pressure sensor 40 and a flow sensor 41 mounted at locations along the breathing system 1 and responsive to gas pressure and flow applied to the patient's airway. The output from the pressure and flow sensors 40 and 41 are supplied to the drive and control unit 30 via cables 42 and 43 respectively. The apparatus also includes an additional position sensor 44 responsive to the position of the piston 21 along the cylinder 22. The sensor 43 could be an optical position sensor or some other position sensor. Alternatively this position information could be derived from signals in the coils.

The external curved surface of the piston 21 forms a sliding seal with the inside of the cylinder 22 so that air or other gas from the conduit 10 is sucked into the cylinder (when the piston moves away from the open end 23) or is forced out of the cylinder (when the piston moves towards the open end). Movement of the piston 21 along the cylinder 22 therefore superimposes an oscillatory waveform on the normal tidal breathing by the patient through the breathing system and does not cause any back pressure on the airway (that is, positive expired pressure PEP or positive applied pressure PAP).

The drive unit 30 is arranged to generate the superimposed oscillatory waveform and this waveform is selected such that it increases movement of mucus within the patient's airways using the outputs of the pressure, flow and position sensors 40, 41 and 44 as necessary. In particular, the waveform is selected to increase the shear forces developed by flow of gas over a thickly lined mucus layer. This is achieved by a combination of an appropriate asymmetric flow pattern and by adjusting frequency, magnitude and phase of the superimposed waveform. Typically, the means for applying the superimposed waveform should be capable of producing oscillations of 0.1 to 20 Hz and with peak flows up to 20 litres/second.

It is believed that an asymmetric waveform may improve mucus clearance, even in the absence of natural clearance mechanisms, such as ciliary beating. In particular, the asymmetric waveform needs to have a higher expiratory peak flow than its inspiratory peak flow so that the mucus is moved primarily in a direction out of the lungs, towards the head, by the gas-liquid interaction. The drive unit 30 monitors the pressure and flow created in the breathing system 1 and adjusts the waveform produced by the actuator 20 to enhance this effect. The effect of the applied waveform is different at different depths of the respiratory system according to the frequency of the applied waveform. The drive unit 30 is arranged to select the frequency of the applied waveform to maximise the therapeutic effect on a particular region of the respiratory system and could be arranged to sweep the frequency across a range so as to vary the region affected.

It is well known that a combination of two different sinusoidal waveforms can produce an asymmetric waveform. FIG. 2 shows two sinusoidal waveforms F1 and F2 of the same amplitude centred about a null position but having different frequencies. The resultant waveform F3 can be seen to be asymmetric with a negative peak that is twice the amplitude of the positive peaks. The drive unit 30 is arranged to manipulate the frequencies and magnitudes of two or more superimposed oscillations to optimise the expiratory peak flow produced and thereby optimise the mucus movement towards the head and out of the lungs.

Various alternative arrangements of electromagnetic actuators are possible. FIG. 3 shows an arrangement where the cylinder 110 includes a piston 111 and nine coils 101 to 109 spaced from one another along the length of the cylinder and alternately wound clockwise and anticlockwise.

FIG. 4 shows an alternative actuator where the coils 201 and 202 are mounted at a fixed position inside the cylinder 203 around the outside of a rod shape permanent magnet 204 supported on the piston 205.

It will be appreciated that the permanent magnet could be mounted in a fixed position and the coils movably mounted on the piston.

1-16. (canceled)

17. Respiratory therapy apparatus including a breathing system arranged to be coupled with a patient's airway, characterised in that the apparatus includes an arrangement coupled with the breathing system that superimposes an oscillatory waveform on normal tidal respiration via the breathing system at an amplitude sufficient to mobilise mucus in the airway and produce a therapeutic effect.

18. Apparatus according to claim 17, characterised in that the waveform is asymmetric and is arranged to produce a greater peak flow in a direction out of the lungs than into the lungs so as to increase flow out of the lungs.

19. Apparatus according to claim 17, characterised in that the waveform is a complex waveform constructed from a series of sinusoidal waveforms of differing periods.

20. Apparatus according to claim 17, characterised in that the arrangement coupled with the breathing system includes a piston movable within a cylinder.

21. Apparatus according to claim 20, characterised in that the arrangement coupled with the breathing system includes a magnetic arrangement for moving the piston in an oscillating manner relative to the cylinder.

22. Apparatus according to claim 21, characterised in that the piston includes a permanent magnet and the cylinder includes an electromagnetic coil arranged to produce a magnetic field within the cylinder that interacts with the field of the permanent magnet to displace the piston along the cylinder.

23. Apparatus according to claim 22, characterised in that the or each electromagnetic coil extends around the outside of the cylinder.

24. Apparatus according to claim 17, characterised in that the apparatus includes a position sensor responsive to the position of the piston.

25. Apparatus according to claim 17, characterised in that the breathing system includes a conduit that is open to atmosphere at one end and opens to a mouthpiece at its opposite end.

26. Apparatus according to claim 17, characterised in that the apparatus includes a pressure sensor responsive to pressure in the breathing system.

27. Apparatus according to claim 17, characterised in that the apparatus includes a flow sensor responsive to flow in the breathing system.

28. Apparatus according to claim 17, characterised in that the apparatus is arranged to adjust the nature of the generated waveform according to feedback from the breathing system.

29. Respiratory therapy apparatus having a conduit with a mouthpiece at one end and open to atmosphere at its opposite end, characterised in that one end of a cylinder opens into the conduit, that the cylinder contains a piston slidable along the cylinder, that the piston carries a permanent magnet that interacts with a magnetic field produced by at least one electromagnetic coil surrounding the cylinder, that the apparatus includes a control unit connected to the at least one coil, that the control unit is connected to receive inputs indicative of the position of the piston in the cylinder and pressure in the conduit, and that the control unit is arranged to cause the piston to oscillate in the cylinder in response to the inputs thereby to superimpose an oscillatory waveform on the normal tidal respiration along the conduit at an amplitude sufficient to mobilize mucus in the patient's airway and produce a therapeutic effect.

30. A method of applying respiratory therapy to a patient's airway, characterised in that an oscillatory waveform is superimposed on normal tidal respiration at an amplitude sufficient to mobilize mucus in the airway and produce a therapeutic effect.

31. A method according to claim 30, characterised in that the waveform is asymmetric and is arranged to produce a greater peak flow in a direction out of the lungs than into the lungs so as to increase flow out of the lungs.

32. A method according to claim 30, characterised in that the waveform is a complex waveform constructed from a series of sinusoidal waveforms of differing periods.

33. A method according to claim 30, characterised in that the method includes moving a piston within a cylinder in an oscillatory manner in an arrangement coupled to a breathing system supplying airflow to the patient's airway to superimpose the oscillatory waveform on the normal tidal respiration.
